(19) United States
(12) Patent Application Publication
(10) Pub. No.: US 2009/0308731 A1
(43) Pub. Date: Dec. 17, 2009

(54) GASIFICATION PROCESS

(76) Inventors: Ernest R. Zabolotny, Burlington, MA (US); Carl E. Tauber, East Greenwich, RI (US); Francis J. Yopak, Southwick, MA (US); James L. Barker, Cape Porpoise, ME (US); Paul G. Lahaye, Kennebunk, ME (US)

Correspondence Address: WOLF GREENFIELD & SACKS, P.C., 600 ATLANTIC AVENUE, BOSTON, MA, 02210-2206, US

(21) Appl. No.: 12/309,956

(22) PCT Filed: Aug. 3, 2007
(86) PCT No.: PCT/US2007/017364

§ 371 Date: Jun. 29, 2009

Related U.S. Application Data
(60) Provisional application No. 60/821,489, filed on Aug. 4, 2006.

(51) Int. Cl. C07C 27/06; B29C 59/14

(52) U.S. Cl. 204/168; 518/702; 518/704

(57) ABSTRACT

The present invention generally relates to gasification processes using high-temperature steam. In one aspect, steam at relatively high temperatures (e.g., at a temperature of at least about 1500° F.) is reacted with a carbon-containing fuel. The carbon-containing fuel may be reacted to produce a carbonaceous intermediate, which may then be reacted to produce a hydrocarbon. The hydrocarbon may then be purified, further reacted, or the like. In some cases, the carbonaceous intermediate may be reacted in a different reactor than the reactor involving reaction of the carbon-containing fuel with the steam. In addition, in some embodiments, H₂ may be produced in reaction systems of the invention, which may be separated and purified, used to produce heat, reacted to produce a hydrocarbon, or the like. In some embodiments, the steam used in this reaction system may be heated to relatively high temperatures using a ceramic heat exchanger, such as those described in U.S. Pat. No. 4,029,465. Other aspects of the invention relate to devices that involve such processes, methods of promoting the making or use of such processes, or the like.

### RELATED APPLICATIONS

This application claims the benefit of U.S. Provisional Patent Application Ser. No. 60/821,489, filed Aug. 4, 2006, entitled “Gasification Process,” by E. R. Zabolotny, et al., incorporated herein by reference.

### FIELD OF INVENTION

The present invention generally relates to gasification processes and, in particular, to gasification processes using high-temperature steam.

### BACKGROUND

The reaction of solid fuels such as coal or coke with steam to produce mixtures of hydrogen and carbon monoxide is highly endothermic (requires the addition of heat) which is typically supplied by burning a portion of the fuel with air or oxygen. The process, which has been in commercial use since the middle of the nineteenth century, began with “town gas” production in large furnaces in which beds of hot coke were alternately blown with air to heat them to incandescence, and then flushed with steam to produce product gas. The furnace output was discharged during the heating cycle or switched to discharge product gas into large gasholders that fed the distribution system for heating and illumination.

The growing chemical process and refinery industries, which demanded carbon monoxide and hydrogen at pressures considerably above atmospheric, drove the development of the pressurized gasifier. The use of air became impractical due to the cost of carrying nitrogen through the process as a diluent, and the potential for the inadvertent production of toxic materials such as hydrogen cyanide. The use of oxygen instead of air eliminated these problems, but lead to the requirement for large air separation plants that were both expensive to purchase and energy intensive in operation. The modern gasifier is the result of evolution in an environment of low to moderate energy prices when emission of carbon dioxide was considered harmless.

### SUMMARY OF THE INVENTION

The present invention generally relates to gasification processes using high-temperature steam. The subject matter of the present invention involves, in some cases, interrelated products, alternative solutions to a particular problem, and/or a plurality of different uses of one or more systems and/or articles.

In one aspect, the invention is a method. In one set of embodiments, the method includes acts of reacting a carbon-containing fuel with steam having a temperature of at least about 1500° F. in a first reactor to produce, at least, H₂ and a carbonaceous intermediate, and reacting the carbonaceous intermediate in a second reactor to produce one or more hydrocarbons.

In another set of embodiments, the method includes acts of reacting a carbon-containing fuel in a first reactor to produce, at least, a carbonaceous intermediate, where at least about 50 mol % of the carbon in the carbon-containing fuel is used to produce the carbonaceous intermediate, reacting the carbonaceous intermediate in a second reactor to produce a hydrocarbon, and at least partially purifying the hydrocarbon produced in the second reactor.

The method, in yet another set of embodiments, includes acts of reacting a carbon-containing fuel with steam having a temperature of at least about 1500° F. to produce, at least, a carbonaceous intermediate, reacting the carbonaceous intermediate to produce a hydrocarbon, and at least partially purifying the hydrocarbon.

In one set of embodiments, the method includes acts of exposing a mixture of a fuel and steam over a ceramic surface to heat the ceramic surface in a reactor, wherein the steam leaving the reactor has a temperature of at least about 1500° F., and at least partially heating the steam prior to entering the reactor using heat extracted from the ceramic surface. The method, in accordance with another set of embodiments, includes an act of exposing a ceramic heat exchanger to steam having a temperature of at least about 1500° F. for a period of time of at least about 1 day.

In accordance with another set of embodiments, the method includes an act of reacting a carbon-containing fuel having no more than about 6.5 wt % hydrogen with steam to produce, at least, H₂ in an amount of at least about 3.5 wt % of the carbon-containing fuel. In still another set of embodiments, the method includes an act of reacting a carbon-containing fuel with steam in a reaction system to produce, at least, H₂, CO and/or CO₂, such that the overall mass ratio of carbon to hydrogen exiting the reaction system is no more than about 50% of the overall mass ratio of carbon to hydrogen in the carbon-containing fuel.

Another aspect of the invention provides an article. In one set of embodiments, the article includes a ceramic heat exchanger, a source of steam in fluidic communication with the heat exchanger, a first reactor in fluidic communication with the ceramic heat exchanger, and a second reactor in solids communication with the first reactor.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate various reaction systems according to certain embodiments of the invention; and

FIGS. 2A-2B illustrate a reaction and a cogeneration system according to another embodiment of the invention.

### DETAILED DESCRIPTION

The present invention generally relates to gasification processes using high-temperature steam. In one aspect, steam at relatively high temperatures (e.g., at a temperature of at least about 1500° F.) is reacted with a carbon-containing fuel. The carbon-containing fuel may be reacted to produce a carbonaceous intermediate, which may then be reacted to produce a hydrocarbon. The hydrocarbon may then be purified, further reacted, or the like. In some cases, the carbonaceous intermediate may be reacted in a different reactor than the reactor involving reaction of the carbon-containing fuel with the steam. In addition, in some embodiments, H₂ may be produced in reaction systems of the invention, which may be separated and purified, used to produce heat, reacted to produce a hydrocarbon, or the like. In some embodiments, the steam used in this reaction system may be heated to relatively high temperatures using a ceramic heat exchanger, such as those described in U.S. Pat. No. 4,029,465. Other aspects of the invention relate to devices that involve such processes, methods of promoting the making or use of such processes, or the like.

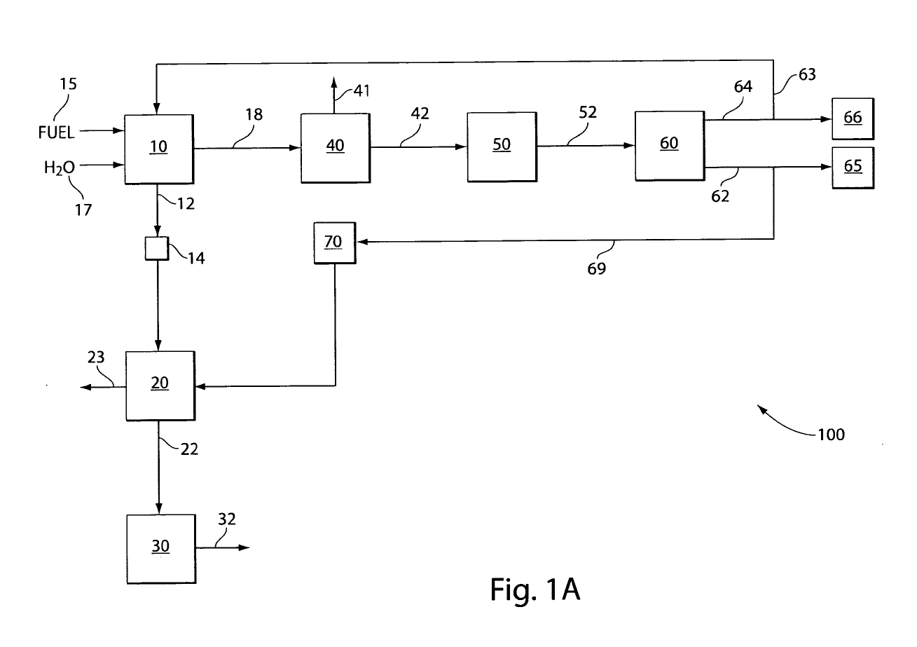

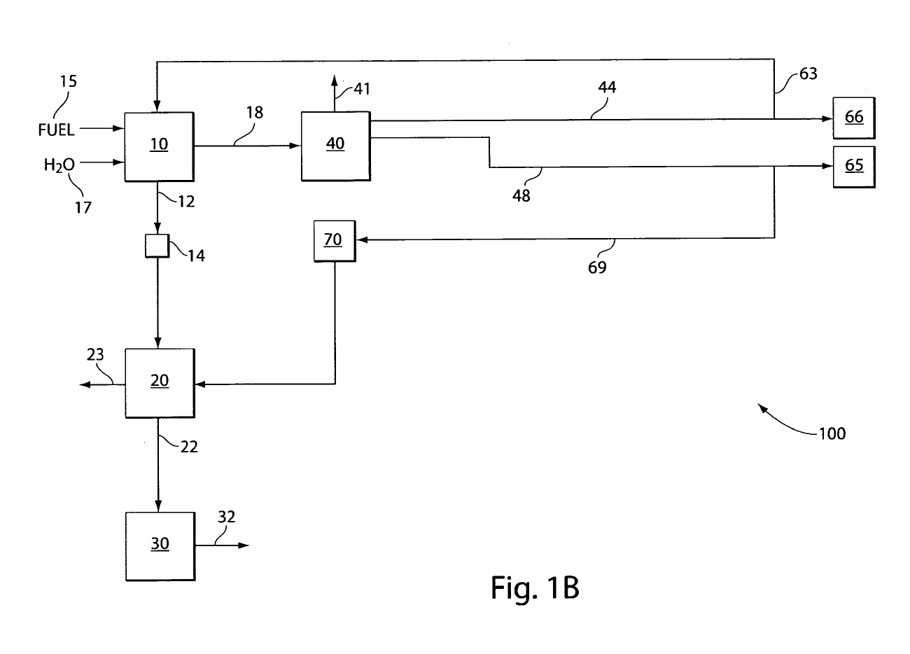

A schematic view of one non-limiting example of a gasification system of the invention is shown in FIG. 1A. In this example, gasification system 100 includes a first reactor 10 and a second reactor 20. A fuel, such as carbon-containing fuel 15, enters first reactor 10. Also entering first reactor 10 is steam (H₂O) 17. The steam and the carbon-containing fuel may be premixed before being introduced into the first reactor, or as is shown in FIG. 1A, the steam and the carbon-containing fuel may be mixed within first reactor 10. In some cases, as discussed below, the steam may be introduced into the first reactor at relatively high temperatures and/or pressures, and in some cases, the steam may be superheated. For example, the steam may be introduced into the reactor at a temperature of at least about 1500° F. or at least about 2000° F. The first reactor itself may also be at a relatively high temperature and/or pressure in some cases. As discussed in detail below, and without wishing to be bound by any theory, it is believed that steam at such relatively high temperatures within the first reactor facilitates reaction of the carbon-containing fuel to produce hydrogen (H₂) and/or carbon monoxide (CO) at relatively greater efficiencies. For example, in some cases, such temperatures may cause relatively less CO₂ to be formed instead of CO.

The production of H₂ and/or CO within the first reactor may also be controlled, for example, by controlling the amount of oxygen entering the first reactor. In some cases, the use of relatively high pressures or temperatures within the first reactor may facilitate the use of lower or no supplemental oxygen within the first reactor. For example, in some instances, a reduction of at least about 30%, about 50%, or about 70% by volume of oxygen may be needed, as compared to reactions in which lower steam temperatures (e.g., around 900 CF to 1000° F.) are used, in order to achieve the same amount of reaction of the fuel to H₂ and/or CO. In certain cases, the oxygen required to oxidize the carbon-containing fuel may be present within the fuel itself, and/or within air contained within the first reactor, and no supplemental oxygen is required.

Of course, depending on the composition of the fuel, other gaseous products may also be formed or contained within the first reactor, for instance, H₂O, H₂S, COS, CO₂, or the like. Gases such as these may exit the first reactor, e.g., via conduit 18 in FIG. 1. In some cases, gases exiting the first reactor may be purified or reacted in some fashion, for example, to remove sulfur (e.g., H₂S, COS, SOₓ, etc.) and/or nitrogen (e.g., NH₃, NOₓ, etc.). There may be one or more such units that can process the gases exiting the first reactor, for example, connected in series or in other configurations.

For instance, as shown in FIG. 1A, gases exiting first reactor via conduit 18 pass into separator 40 (e.g., a desulfurization scrubber, where sulfur-containing species such as H₂S or COS are removed, e.g., by converting the sulfur into CaSO₃, elemental sulfur, H₂SO₄, or the like, optionally exiting via conduit 41). Scrubbers or other operations able to remove species such as sulfur, nitrogen, CO₂, or the like are known by those of ordinary skill in the art.

As another example, H₂ exiting the first reactor may be purified and used within the reactor, stored for future use, stored as a product, or the like. In some cases, the amount of H₂ within the exiting gas may be increased, for instance, via a water gas shift reaction, where CO and H₂O can be converted to form CO₂ and H₂. In some cases, the water gas shift reaction may be facilitated by the use of catalysts such as Fe₃O₄ (magnetite), or other transition metals or transition metal oxides. As an example, referring again to FIG. 1A, gases exiting separator 40 via conduit 42 may be directed into unit 50, which may be a water gas shift reactor. From there, the amount or concentration of H₂ present within the exiting gases may be increased via the water gas shift reaction or other such reactions. As another example, in some cases, gases exiting the separator may be relatively rich in hydrogen, and thus, the gas may be directed to processes using hydrogen. For instance, as is shown in FIG. 1B, gases rich in hydrogen may exit separator 40 via conduit 48 to be collected within collector 65, and/or used for heating or other reactions within system 100 and/or within other systems, etc., while the other gases may exit separator 40 via conduit 44 and can be recycled via conduit 63, or captured or sequestered, e.g., for long-term storage, as indicated by unit 66.

Referring again to unit 50 in FIG. 1A, gases exiting unit 50 via conduit 52 may contain H₂ and/or other gases. In some cases, the gas may be purified or otherwise processed in some fashion. For instance, as is illustrated in FIG. 1A, conduit 52 may be directed to unit 60, which may be, for example, a hydrogen purification unit. The hydrogen purification unit may be, for example, a membrane separator, for instance, utilizing a hydrogen separation membrane. As shown in FIG. 1A, from unit 60, a stream of purified hydrogen (or at least, a gas enriched in hydrogen) exits unit 60 via conduit 62, and the remaining gases (e.g., containing CO₂, H₂O, or the like) exit via conduit 64. The hydrogen stream in conduit 62 may be collected as a product (e.g., to be used as a fuel, as is shown by collector 65 in FIG. 1), reacted to produce heat (and H₂O) for heating (e.g., for heating the steam entering first reactor, not shown in FIG. 1), used in other reactors or unit operations (not shown in FIG. 1), and/or used for further reaction within reactor 50 via conduit 69, e.g., as is discussed below.

The remaining gases exiting the hydrogen separator may be disposed of by any suitable technique. For example, some or all of the CO and/or CO₂ may be recycled back to the first reactor, e.g., via conduit 63 as is shown in FIG. 1, and/or the CO and/or CO₂ (and/or other gases) may be captured or sequestered, e.g., for long-term storage, as indicated by unit 66. Similarly, water or other gases within the exiting gas stream may be removed for disposal, processed or purified, using in other reactions, or the like.

In some embodiments of the invention, the first reactor is run under operating conditions such that not all of the carbon-containing fuel is fully oxidized to form CO and/or CO₂, but instead, a portion of the carbon-containing fuel may be reacted to form a carbonaceous intermediate. In some cases, the composition of the carbonaceous intermediate is not precisely known, and can simply be referred to as a “char” or an “ash.” The carbonaceous intermediate may then be reacted to produce one or more hydrocarbons, for example, in a second reactor, such as second reactor 20 in FIG. 1. Waste material produced in second reactor 20 may then exit second reactor 20 through conduit 23. Accordingly, the carbonaceous intermediates produced within first reactor 10 may thus pass via conduit 12 to second reactor 20. The first and second reactors may be run at the same or different temperatures and/or pressures. For instance, as is shown in FIG. 1, first reactor 10 and second reactor 20 are separated by a gas-sealing interlock 14, which allows the reactors to be run at different pressures. Thus, for instance, solids or liquids may be transferred from the first reactor to the second reactor, i.e., there may be solids or liquid communication between the first and second reactors, while the amount of gaseous transfer that occurs between the first and second reactors is limited.

Within the second reactor, the carbonaceous intermediate may be reacted to produce one or more hydrocarbons (or other carbon-containing products), for instance, methane (CH₄) or a polymer. The hydrocarbons may then be removed from the second reactor through conduit 22, and optionally, purified using purification unit 30, before exiting via conduit 32. In some cases, hydrogen gas may be introduced into the second reactor to facilitate the reaction, and in certain embodiments, at least a portion of this hydrogen may arise from other reactions within the reaction system (e.g., delivered by via conduit 69 from hydrogen separator 60, as is shown in FIG. 1). In some cases, the hydrogen may be ionized via an optional plasma generator (e.g., unit 70 in FIG. 1), which may increase the reactivity of the hydrogen for use within the second reactor.

As discussed above, in one aspect, the gasification system includes a first reactor able to react high-temperature steam and a carbon-containing fuel to produce gases such as H₂, CO, CO₂, H₂O, etc., and/or one or more carbonaceous intermediates. In some cases, the conditions of the first reactor are chosen or optimized such that volatiles and/or reactive carbon within the fuel may react with the steam to produce H₂ and/or CO.

Any suitable carbon-containing fuel may be used, for example, a fossil fuel, or biomass. Non-limiting examples of fossil fuels include oil, gas, peat, coke, graphite, or coal, which may contain any amount or concentration of impurities such as sulfur. For instance, if coal is used as a fuel, the coal may be lignite, subbituminous, bituminous, anthracite, etc., and the coal may have a relatively high sulfur content (e.g., greater than about 1% or 2% by weight), a relatively high hydrogen content, or a relatively high oxygen content, etc. Hydrogen present within the coal may be reacted to form H₂ and/or H₂O, while oxygen within the coal may be reacted to form CO and/or CO₂, depending on the reaction conditions. Similarly, sulfur within the coal may be reacted to form H₂S, COS, etc. Such gases may be removed and purified, and stored, released, sequestered (i.e., prevented from being released into the atmosphere, for example, by storage in the ocean, or underground in oil fields, saline aquifers, coal seams, etc.), and/or used in other reactions, as described herein.

In some cases, the carbon-containing fuel may contain biomass. The biomass may be any material derived from a living organism, often plants (e.g., trees, grain, etc.), and may be purified or, in some cases, used without purification. For example, plants and/or plant residues such as corn, sugar cane, sugar beets, straw, rice, cotton, switchgrass, hemp, poplar, willow, bamboo, miscanthus, bagasse, etc., may be harvested, optionally out or ground into smaller pieces, and used in a fermentation reaction with or without fisher processing. The biomass within the first reactor may be reacted with high-temperature steam, which may facilitate breakdown of the carbon-containing fuel into small compounds.

In some embodiments, the reaction within the first reactor may not be run to completion and full combustion of the carbon-containing fuel, and some of the carbonaceous intermediates may be removed from the first reaction, for example, as solid particulates. In some cases, as discussed below, the carbonaceous intermediates may be further reacted, e.g., in a second reactor, to produce one or more hydrocarbons or other carbon-containing products. Thus, for example, at least about 20 mol %, at least about 30 mol %, at least about 40 mol %, at least about 50 mol %, at least about 60 mol %, at least about 70 mol %, at least about 80 mol %, or at least about 90 mol % of the carbon contained within the carbon-containing fuel may be used to produce the carbonaceous intermediate.

The composition of the carbonaceous intermediate so produced may not be precisely known, and may be a mixture of various carbon-containing species. Often, the carbonaceous intermediates will be collected as a solid or a liquid within the first reactor, e.g., as a “char” or an “ash,” depending on the composition of the fuel. As other examples, the carbonaceous intermediate may be present as solid particulates (e.g., soot), a liquid, a sludge, or the like. In some embodiments, the carbonaceous intermediate may have a relatively high amount of carbon present, relative to the composition of the carbon-containing fuel, as some of the hydrogen and/or oxygen present within the fuel may have been released as H₂, H₂O, CO, CO₂, etc. Other species besides carbon-containing compounds may be present in the carbonaceous intermediate as well, for instance, nitrates, silicates, metals, minerals, oxides, or the like. The carbonaceous intermediate may be present in various forms, depending on the reaction occurring within the first reactor.

In some cases, the first reactor is run under conditions in which no supplemental oxygen is added, or relatively low amounts of oxygen are added. Oxygen necessary for reaction within the first reactor to occur may be provided by the fuel and/or air within the first reactor. Thus, for example, the first reactor may contain no more than about 21 vol % O₂, no more than about 20 vol % O₂, no more than about 15 vol % O₂, etc. In other embodiments, however, some supplemental amount of oxygen may be added, for example, as a pure stream of O₂, as a mixture containing oxygen, as an oxygenated fuel, or the like. For example, there may be a 50% to 70% by volume reduction of supplemental oxygen used by this gasification process, relative to other, similar gasification processes.

In one embodiment, a carbon-containing fuel having no more than about 3 wt %, about 4 wt %, about 5 wt %, or about 6.5 wt % hydrogen may be reacted with steam to produce H₂ in an amount of at least about 3.5 wt %, at least about 5 wt %, at least about 7 wt %, or at least about 9.2 wt % of the carbon-containing fuel. In some cases, the overall CO₂ (including the gasification process, auxiliary processes, power sources, etc.) produced by a gasification system of the invention may be in an amount no more than about 13, about 15, about 17, or about 19 times the mass of H₂ produced. In one set of embodiments, the reaction may occur such that the overall mass ratio of carbon to hydrogen exiting the reaction system is no more than about 50%, about 40%, or about 30% of the overall mass ratio of carbon to hydrogen in the carbon-containing fuel.

The first reactor may be any suitable reactor able to react the carbon-containing fuel and the steam. For example, if the carbon-containing fuel is solid, any suitable solid-gas reactor may be used, for example, a fluidized bed reactor, a fixed bed reactor, an entrained flow reactor, or the like. In some cases, the carbon-containing fuel may be pulverized, crushed, or otherwise converted into particulate matter prior to reaction in the first reactor. Similarly, if the carbon-containing fuel is a liquid, any suitable liquid-gas reactor may be used, for example, a packed tower, a spray tower, a wetted-wall tower, a tray tower, or the like.

The reaction within the first reactor may be run at relatively high temperatures and/or pressures, and thus, the first reactor may be a pressure vessel and/or a vessel able to withstand relatively high temperatures. The reactor may thus be constructed out of any suitable materials able to withstand such temperatures or pressures, such as stainless steel. For instance, the pressure within the first reactor may be at least about 5 atm, at least about 10 atm, at least about 15 atm, at least about 20 atm, or more in some cases. The first reactor may be pressurized and the pressure may be controlled, using any suitable technique known to those of ordinary skill in the art. For example, the incoming fuel and/or steam may be pressurized, and/or additional gases added to the first reactor in order to reach a desired pressure. Sensors, controllers, and the like may be used to control the pressure within the reactor, and the use of such sensors and controllers for process control is understood by those of ordinary skill in the art.

The temperature within the first reactor may also be relatively high. For instance, the reaction within the first reactor may occur at relatively high temperatures of at least about 1500° F. (about 816° C.), at least about 2000° F. (about 1093° C.), at least about 2500° F. (about 1371° C.), at least about 3000° F. (about 1649° C.), or at least about 3500° F. (about 1927° C.), etc. In some cases, as discussed below, the steam entering the first reactor may be at such temperatures, and the entering steam may thus be used to heat the first reactor. Of course, in other embodiments, other methods may be used to heat the first reactor, for example, by heating the incoming fuel, by heating the first reactor itself, etc. In still other embodiments, a combination of techniques may be used. In addition, the first reactor may be thermally insulated. In addition, the temperature within the first reactor may be controlled using any suitable technique. For example, the incoming fuel and/or steam may be heated (e.g., as discussed below). Sensors, controllers, and the like may also be used to control the temperature within the reactor, and the use of such sensors and controllers is understood by those of ordinary skill in the art. In certain embodiments, material exiting the first reactor may be at relatively high temperatures, and a heat exchanger may be used to cool this material, as discussed below.

As mentioned, the steam may be introduced into the reactor at relatively high temperatures, according to certain aspects of the invention. In some cases, other gases besides steam may be introduced as well, for example, air. The steam may be heated using any suitable technique, and in some cases, the steam may be produced within the gasification system without requiring additional energy inputs, e.g., the energy used to heat the steam may be derived from the carbon-containing fuel. For instance, as discussed below, in one embodiment, the steam is heated by oxidizing hydrogen produced in the first reactor, and using at least a portion of that heat to at least partially heat the steam. In some embodiments, a heat exchanger, such as a ceramic heat exchanger, may be used to heat the steam to such temperatures. The heat exchanger may have any suitable configuration, for example, a shell-and-tube heat exchanger or a plate heat exchanger. In some cases, the ceramic heat exchanger may be stably used under such conditions for relatively long periods of time, for example, at least about 12 hours, at least about 1 day, at least about 1 week, at least about 4 weeks, at least about 1 year, at least about 2 years, at least about 5 years, etc.

Non-limiting examples of such ceramic heat exchanges are known in the art, for example, as disclosed in U.S. Pat. No. 4,060,379, issued Nov. 29, 1977, entitled “Energy Conserving Process Furnace System and Components Thereof,” by LaHaye, et al.; U.S. Pat. No. 4,134,449, issued Jan. 16, 1979, entitled “Bellows Sealing Arrangement,” by La Haye, et al.; U.S. Pat. No. 4,332,295, issued Jun. 1, 1982, entitled “Composite Ceramic Heat Exchange Tube,” by LaHaye, et al.; U.S. Pat. No. 4,437,217, issued Mar. 20, 1984, entitled “Composite Ceramic Heat Exchange Tube,” by Lallaye, et al.; U.S. Pat. No. 5,765,596, issued Jun. 16, 1998, entitled “Ceramic Heat Exchanger,” by LaHaye, et al.; or a U.S. Department of Energy Report, Contract Nos. DE-AC21-94 MC30327 and DE AC21-93 MC30318, entitled “High Pressure Coal-Fired Ceramic Air Heater.”

In one non-limiting embodiment, the ceramic heat exchanger includes a main tube of a high temperature resistant material, which may be constructed of a porous thermal shock resistant ceramic outer shell and an inner tube. The tubes may be provided in one or more sections including two sealed ends. In some cases, the inner tube, the seals and the outer shock resistant containing shell may be thermally and chemically compatible throughout the operating range of the heat exchanger. The inner tube may be concentrically disposed within the main tube and the end to form a sealing joint at opposite ends of the tube to mate with matching tubes or components. The ends may be capable of being highly polished to form a high pressure sealing surface. The ends of the ceramic tube typically seal to an adjacent like tube or an end adapter such as described in U.S. Pat. No. 4,060,379. The inner tube may be constructed of a more dense material than the main tube and can be formed to be less porous than the main tube to provide a surface which can be polished. The inner tube may also act as a containment vessel for a high pressure gas such as steam that has small molecules which can readily penetrate porous walls. The composite tube may be optimized geometrically for gas heat transfer. For instance, the tube may display the characteristic of being capable of containing high pressure gases and yet will also be able to stand thermal shock or corrosion. In some cases, the inner tube or liner can be constructed of a ceramic material having a density of more than 85% by volume of full density. The main tube may have a density of at least 80% of full density, and in some cases, the density may be less than the density of the inner liner or seals. The liner and the end seals may be of substantially the same density, or of different densities. In some cases, the end seals can be of greater density than the inner liner, as the end seals may require polishing to a surface finish of 4 to 65 micro-inches roughness height to provide a seal to contain the high pressure gas.

The inner tube or liner may be constructed of silicon carbide, silicon nitride or other suitable ceramic materials. The silicon carbide may be selected to have a density of, for instance, on the order of 3.0 g/cm³. Where silicon nitride is used, it may have a density on the order of 3.0 g/cm³. The inner tube or liner, in some cases, can have a density greater than 85% by volume of theoretical full density. For some applications, it is possible that the inner liner can also be constructed of metal having a thermal coefficient of expansion which is compatible with the ceramic material in the outer shell.

In some embodiments, there is also provided a method of fabricating a composite heat exchange tube having a porous thermal shock resistance ceramic main tube for the passage internally of heated gas or air at temperatures of ambient temperatures (about 77° F.) to at least about 1500° F. or 2000° F., or at least between about 900° F. and about 1500° F. or about 2000° F. Ceramic end inserts can be provided which can form a seal at each end of the heat exchange tube and which may be made of a more dense ceramic material than the main tube. These inserts may be disposed in respective ends of the main tube and there may then be a thin ceramic liner deposited within the main tube. The liner may be substantially impervious to fluids flowing within the tube at pressures at least up to 500 psi. The liner can be formed in many different ways, such as by the use of vapor deposition, either physical or chemical, a glazing operation, plating, sputtering, flame spraying, or electrostatic deposition. Furthermore, the liner may be formed separately and later inserted into the main tube in some cases.

The heat exchange tube may be made of ceramic, and the ceramic may be chosen to provide resistance to high temperatures encountered in the gas stream, e.g., from ambient temperatures to temperatures of over 3500° F. (or other temperatures as described herein), depending on the particular application, at the same time providing for good heat transfer. Examples of suitable ceramics include, but are not limited to, silicon carbide or silicon nitride. Other ceramics can be used, for instance, ceramics having a thermal conductivity of at least 3 BTU/hr/ft²/° F./ft. For example, the main tube may be cast from a commercially available castable silicon carbide such as Carbofrax 11, a product of the Carborundum Company of Niagara Falls, N.Y. This product is typically mixed with water and cast to a desired shape and then fired to temperatures over 1800° F. to develop strength and good thermal conductivity. The castable silicon carbide may also use a material such as calcium-aluminate as a binder. This silicon carbide material may, for example, be cast at room temperature and allowed to cure at room temperature. It may then be preheated for a period of time and then fired to a suitable temperature, e.g., 2100° F., for a period of hours.

In some aspects, carbonaceous intermediates from the first reactor may be reacted to produce one or more hydrocarbons or other carbon-containing products, for example, in a second reactor. In some cases, the hydrocarbons contain only hydrogen and carbon (e.g., methane, CH₄, ethane, CH₃CH₃, etc.), or in some cases, the hydrocarbons may contain one or more heteroatoms such as oxygen (e.g., as in methanol, CH₃OH or ethanol, CH₃CH₂OH).

The carbonaceous intermediates may be reacted to produce lower hydrocarbon species, such as CH₄, CH₃CH₃, or the like. Other examples include the production of alkanes, alcohols, organic acids, anhydrides, etc. In some embodiments, hydrogen or hydrogen plasma, as discussed below, may be reacted with the carbonaceous intermediates. In certain cases, a portion of the hydrocarbons or other carbon-containing products may be purified (e.g., using a scrubber, a distiller, an extractor, or other operations known to those of ordinary skill in the art), and collected as a product and/or for sequestration, reacted to produce heat for heating (e.g., for heating the steam entering first reactor), used in other reactors or unit operations, or the like, as well as any combinations of these and/or other operations. In some cases, at least a portion of the hydrocarbons or other carbon-containing products that are produced may be sequestered or stored.

As mentioned, in some embodiments, the carbonaceous intermediates may be reacted to produce one or more hydrocarbons or other carbon-containing products in a second reactor. Without wishing to be bound by any theory, it is believed that the reaction of the carbonaceous intermediates and the hydrogen (each of which may have been generated in the first reactor) to produce a product in a second reactor (which may be thermally isolated from the first reactor) reduces the heating and energy costs associated with heating and maintaining a carbon-containing fuel at the relatively high temperatures in the first reactor, as previously described (e.g., at temperatures of at least about 1500° F.). Accordingly, the second reactor may be at a temperature lower than the first reactor. In addition, heat may be recovered from the first reactor, as discussed below, prior to the reaction of the carbonaceous intermediates and the hydrogen at a lower temperature in the second reactor. It is believed that such an approach represents a significant advance over the state of the art.

The second reactor may be any suitable reactor able to react the carbonaceous intermediate, e.g., with hydrogen, oxygen, or the like. For example, if the carbonaceous intermediate is solid, any suitable solid-gas reactor may be used, for example, a fluidized bed reactor, a fixed bed reactor, an entrained flow reactor, or the like. In some cases, the carbonaceous intermediate may be pulverized, crushed, or otherwise converted into particulate matter prior to reaction in the second reactor.

The second reactor may be run at any suitable temperature and pressure, and the reactor may be constructed out of any suitable materials able to withstand such temperatures or pressures, such as stainless steel. The temperatures and pressures within the first reactor may or may not be the same as in the first reactor. Thus, in some cases, the first reactor may be substantially thermally isolated from the second reactor, and/or the first reactor and the second reactor may be separated by a gas-sealing interlock, allowing the reactors to operate at different pressures. Techniques for thermally isolating reactors, and gas-sealing interlock devices will be known to those of ordinary skill in the art.

In some embodiments, the pressure within the second reactor may be at least about 5 atm, at least about 10 atm, at least about 15 atm, at least about 20 atm, or more in some cases. Thus, in some embodiments, the second reactor is a high-pressure vessel. The second reactor may be pressurized, and controlled, using any suitable technique. Sensors, controllers, and the like may be used to control the pressure within the second reactor, and the use of such sensors and controllers is understood by those of ordinary skill in the art. Similarly, the temperature within the second reactor may be, in some embodiments, at least about 500° F. (about 260° C.), at least about 600° F. (about 316° C.), at least about 700° F. (about 371° C.), at least about 800° F. (about 427° C.), at least about 900° F. (about 482° C.), or at least about 1000° F. (about 538° C.). However, in one embodiment, the temperature of the second reactor is less than about 1500° F. (about 816° C.), less than about 1200° F. (about 649° C.), or less than about 1000° F. (about 538° C.). Sensors, controllers, and the like may also be used to control the temperature within the first reactor, and the use of such sensors and controllers is understood by those of ordinary skill in the art. In certain embodiments, material exiting the second reactor may be at relatively high temperatures, and a heat exchanger may be used to cool this material.

In another aspect, however, the carbonaceous intermediates are not reacted to produce one or more hydrocarbons, but instead may be directly removed, optionally purified, and stored for later use, sequestered, or the like. For instance, in one embodiment, carbonaceous intermediates such as solid particulates (e.g., soot), a liquid, a sludge; an ash, a char, or the like are removed from the first reactor, using any suitable technique, and are sequestered (e.g., buried) as a method of removing carbon from the atmosphere.

In one aspect of the present invention, gases exiting the first reactor may be purified or reacted in some fashion, for example, to remove sulfur (e.g., H₂S, COS, SOₓ, etc.) and/or nitrogen (e.g., NH₃, NOₓ, etc.) from the exiting gases. There may be one or more such units that can process the gases exiting the first reactor, for example, connected in series or in other configurations, depending on the particular application. In certain embodiments, gases exiting the first reactor may be at relatively high temperatures, and a heat exchanger may be used to cool the gases, as discussed below. The exiting gases may be directed to one or more units able to remove one or more species from the exiting gas, for instance, to comply with environmental regulations, to isolate or purify one or more desired species, etc. Those of ordinary skill in the art will be aware of various techniques to separate gases, for example, via membrane separation.

As an example, in one set of embodiments, the gases may be passed to a desulfurization unit able to remove sulfur-containing species such as H₂S, COS, or SOₓ. For instance, the desulfurization unit may be a desulfurization scrubber which is able to remove sulfur-containing species through the conversion of CaCO₃ into CaSO₃, MgCO₃ into MgSO₃, etc. In another embodiment, the sulfur-containing species may be converted to form elemental sulfur and/or sulfuric acid (H₂SO₄), which may be collected and subsequently used or sold. In some cases, sulfur can be removed by reduction of sulfur-containing species to hydrogen sulfide (H₂S), which is then removed by a scrubbing with a suitable solvent. The solvent can be regenerated in some cases by heating to drive out the hydrogen sulfide. Various desulfurization processes are commercially available, and can be used here. In certain cases, the hydrogen sulfide can be converted to elemental sulfur, or oxidation to from sulfur trioxide to make sulfuric acid, and these processes are also commercially available.

As another example, the gases may be passed into a nitrogen-removal unit. For instance, NOₓ within the exiting gases may be decomposed to nitrogen and water vapor using ammonia (NH₃) using a suitable catalyst, trapped via a NOₓ adsorber or a NOₓ trap, or the like.

In another set of embodiments, hydrogen within the exiting gases may be purified or isolated, and/or the exiting gases may be treated to increase the hydrogen concentration. Hydrogen may be separated from the other exiting gases using techniques known to those of ordinary skill in the art, for example, using a hydrogen separation membrane such as a ceramic membrane, a microporous membrane, or a palladium membrane. Another example is Pressure Swing Adsorption (PSA) into a suitable solid absorbing material. In another example, if hydrogen is to be separated from a gas such as carbon dioxide which has an acid reaction, the hydrogen may be scrubbed with a suitable alkaline solvent. In some cases, the adsorbent or solvent can be regenerated and reused. Many of these processes are commercially available.

For instance, in some cases, the exiting gases are subjected to a unit which causes the water gas shift reaction to occur with any CO and H₂O present within the exiting gases to produce CO₂ and H₂, i.e.:

CO+H₂O→CO₂+H₂

In some cases, the water gas shift reaction may be facilitated by the use of catalysts such as Fe₃O₄ (magnetite), or other transition metals or transition metal oxides. Various water gas shift reaction processes are commercially available. The hydrogen may then be purified to form a product stream, as discussed above.

Hydrogen produced using any of the techniques discussed above (e.g., via separation and/or via the water gas shift reaction) may be used for a number of different applications, according to various aspects of the invention. For example, in one set of embodiments, hydrogen gas may be collected, optionally purified, and stored for later use, for example, to produce energy, e.g., within a fuel cell. Techniques for collecting and storing hydrogen gas, e.g., under pressure, are known to those of ordinary skill in the art.

In another set of embodiments, the hydrogen gas may be reacted to produce heat, e.g., by combination with oxygen to produce water. The heat may be used for various applications, for example, to heat steam and/or fuel entering the first reactor, and/or to heat other processes or reactions. As a specific example, the hydrogen may be sent to an afterburner, where the hydrogen is reacted with oxygen to produce heat and water. The afterburner may be in thermal communication (e.g., via a heat exchanger), for example, with steam entering the first reactor, thereby allowing the transfer of the heat produced from the hydrogen reaction to at least partially heat the steam.

In yet another set of embodiments, the hydrogen gas may be used in other reactors or unit operations, e.g., within the system described herein, and/or in other systems. For example, the hydrogen may be directed to the second reactor, e.g., for reaction with a carbonaceous intermediate. In some cases, the hydrogen may be purified or otherwise processed prior to introduction into the second reactor. For instance, in one embodiment, the hydrogen may be directed to a plasma generator, which may increase the reactivity of the hydrogen for use within the second reactor, e.g., by converting at least a portion of the hydrogen into a plasma and/or into ionized hydrogen. In some cases, the plasma may be generated by applying high voltages to the hydrogen. In some cases, the plasma generator may be run using electric fields that are at least sufficient to ionize at least a portion of the hydrogen. In some cases, relatively lower voltages may be used to minimize energy expenditures. For example, the voltage may be at least about 6 V, at least about 10 V, or at least about 30 V. In some cases, the voltage may be no more than about 50 V, about 100 V, etc., although higher voltages may be initially used to start the process. Examples of such plasma generators include, but are not limited to, plasma torches for welding or cutting.

In still another set of embodiments, the hydrogen may be used in any combination of the above applications and/or within other applications. For example, a first portion of the hydrogen may be reacted to produce heat, a second portion may be reacted within the second reactor, and a third portion may be collected and stored, e.g., as an energy source. Thus, in general, certain embodiments of the invention allow for the reaction of a fuel to produce hydrogen for various uses and applications.

In some aspects of the invention, CO and/or CO is produced. For example, combustion of the carbon-containing fuel within the first reactor may produce CO and/or CO₂, and additional CO and/or CO₂ may be produced in the water gas shift reaction, as discussed above. In some cases, such gases are released to the atmosphere, but in other embodiments, some or an of the CO and/or CO₂ may be captured and stored or sequestered, e.g., limiting or eliminating the CO and/or CO₂ released to the atmosphere. Accordingly, in some embodiments, at least a portion of the CO and/or the CO₂ produced in the first reactor and/or in the second reactor may be stored or sequestered. In some embodiments, the CO (e.g., produced in the first reactor) may be oxidized to form CO₂, and/or or reduced in some fashion, e.g., to produce a hydrocarbon such as methane, an alcohol such as methanol or ethanol, or the like. An example is the water gas shift reaction, as described above.

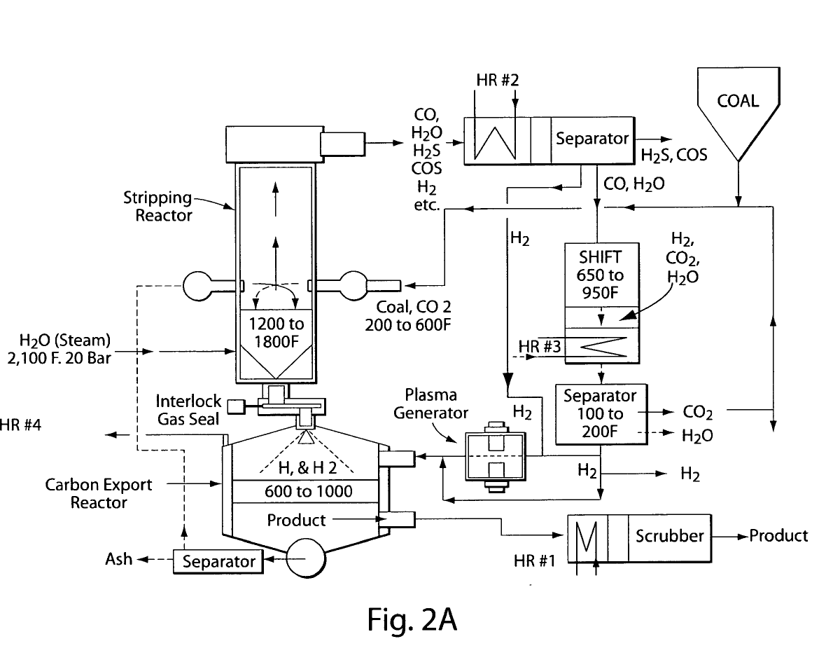

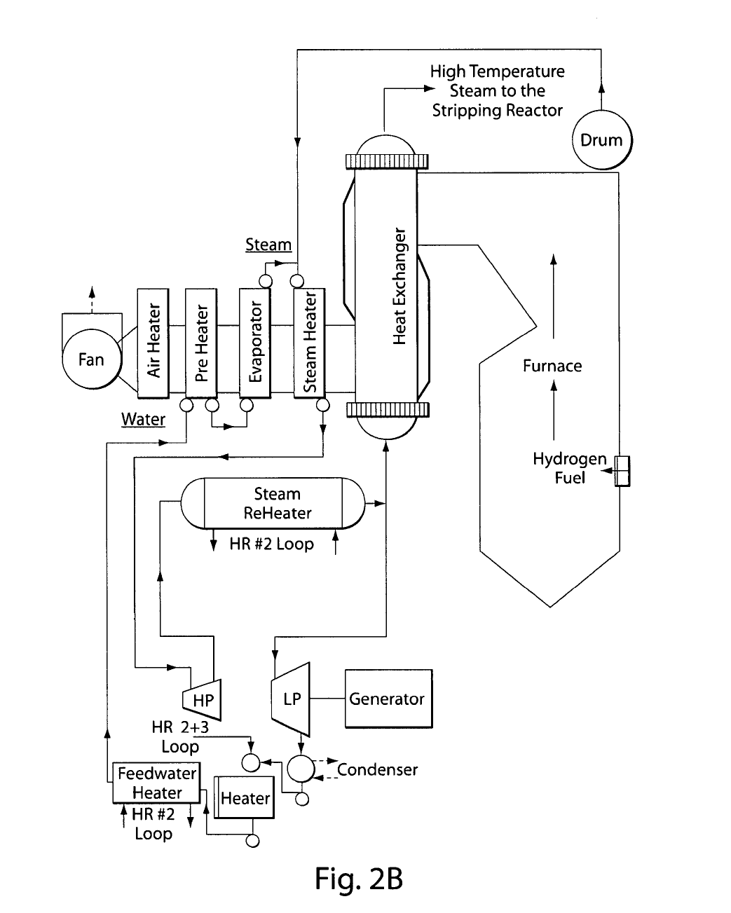

In one aspect of the invention, one or more heat exchangers may be used to efficiently use heat or energy within the reaction system. For example, if the first reactor is run at a higher temperature than the second reactor, a heat exchanger may be used to recover some of the heat and otherwise used, for example, to heat steam entering the first reactor. Those of ordinary skill in the art will be familiar with the design of heat exchange networks to efficiently use heat in a reaction system. Other, non-limiting examples where heat may be recovered include the gases exiting the first reaction (e.g., in a separator or in a water shift gas reactor), or hydrocarbons or other carbon-containing products exiting the second reactor. Some of these heat exchangers have been indicated in FIG. 2A as a non-limiting example.

The following examples are intended to illustrate certain embodiments of the present invention, but do not exemplify the full scope of the invention.

### Example 1

This example describes a two-reactor scheme for extraction of maximum economic value from a wide variety of fuels with little or no emission of greenhouse gases. In this example, a carbon-containing fuel and high temperature steam are mixed in a first “stripping” reactor to convert volatiles or reactive carbon to hydrogen and carbon monoxide. Unreacted char (or other carbonaceous intermediates) produced in this reaction is discharged from the stripping reactor to a second “carbon export reactor,” in which the char is reacted with hydrogen to form economically valuable hydrocarbons (or other carbon-containing products). Waste carbon can be sequestered as carbon dioxide or solid carbon. Hydrogen (e.g., produced as described above) may be burned to power this process, in some cases. Ash, sulfur, or other residues can also be separated and converted to forms having good economic value or low disposal costs.

The process described in this example is shown schematically in FIG. 2. As shown in FIG. 2A, this process includes two reactors. The first reactor is called the stripping reactor, or stripper, and the second reactor is called the carbon export reactor. Both reactors are substantially thermally insulated from the surroundings and from each other, and may be pressure vessels, e.g., that are rated for operation at pressures of 20 atm to 70 atm. An interlock, gas-sealing device may allow the ability to transfer carbonaceous intermediates from the stripper to the carbon export reactor, e.g., as a solid or a liquid residue, etc. This interlock may also be used to prevent or at least minimize mixing of gases between the two reactors, which may operate at different pressures and/or temperatures. Also shown in FIG. 2A are examples of potentially suitable process temperatures and pressures.

The stripping reactor is an entrained flow reactor in this example, but other reactors, such as a fluidized bed or a fixed bed reactor, are also possible, for instance, if a specific fuel should make either of these designs desirable. The stripping reactor can be used to mix a fuel with high temperature steam under conditions that may cause volatiles or the reactive carbon within the fuel to react with the steam, e.g., to form hydrogen and carbon monoxide. The stripping reactor may be run at any suitable temperature, for example, at or above about 1500° F. or above about 2000° F.

Heat energy needed to drive the formation of hydrogen and carbon monoxide in the first reactor, other than sensible energy present in the fuel, may be supplied in some cases by a cogeneration plant. A non-limiting example of a cogeneration plant is shown in FIG. 2B. The heat energy needed to drive the reactions in the first reactor may be present in the steam, and may be provided by the cogeneration plant. The cogeneration plant may heat the steam to temperatures in excess of about 1500° F. or about 2000° F., for instance, using a ceramic heat exchanger such as those described in U.S. Pat. No. 4,060,379, issued Nov. 29, 1977, entitled “Energy Conserving Process Furnace System and Components Thereof,” by LaHaye, et al.; U.S. Pat. No. 4,134,449, issued Jan. 16, 1979, entitled “Bellows Sealing Arrangement,” by La Haye, et al.; U.S. Pat. No. 4,332,295, issued Jun. 1, 1982, entitled “Composite Ceramic Heat Exchange Tube,” by LaHaye, et al.; U.S. Pat. No. 4,437,217, issued Mar. 20, 1984, entitled “Composite Ceramic Heat Exchange Tube,” by Lallaye, et al.; U.S. Pat. No. 5,765,596, issued Jun. 16, 1998, entitled “Ceramic Heat Exchanger,” by LaHaye, et al.; or a U.S. Department of Energy Report, Contract Nos. DE-AC21-94 MC30327 and DE AC21-93 MC30318, entitled “High Pressure Coal-Fired Ceramic Air Heater.”

The thermal energy in the steam (or the temperature of the steam) may be augmented and controlled, in some embodiments, by an afterburner combusting a portion of the hydrogen produced as described above with oxygen, for instance, at the exit of the heat exchanger. In some cases, the ceramic heat exchanger and the hydrogen afterburner can raise the process steam temperature up from an inlet temperature of about 950° F. to more than about 1500° F., for example, about 2000° F. or about 3600° F. A conventional power plant steam system reheater may be used to deliver steam to the heat exchanger at a temperature of 950° F. to 1000° F. or 1100° F., as shown in FIG. 2B. Such conventional steam generation systems are known to those of ordinary skill in the art.

Also shown in FIG. 2A are various heat recovery points, labeled as HR #1, HR #2, HR #3, and HR #4. These heat recovery points are shown by way of example only, and can be varied by those of ordinary skill in the art using no more than routine optimization, depending on factors such as the temperatures of the various units, the type of fuel used, the spatial configuration of the reactor, etc. A portion of this heat may be is used to offset a portion of the cogeneration plant fuel consumption (e.g., consumption of H₂ produced as described above), due to heat transfer to the incoming steam. Systems suitable for transferring heat between operations within a reaction system (e.g., via various heat exchangers) will be known by those of ordinary skill in the art.

Complete reaction of the carbon-containing fuel in the first reactor is not necessarily required. Instead, carbonaceous intermediates may be created and transferred to the carbon export reactor. The carbonaceous intermediates may contain carbon that can be converted to gas or liquid, forming a product for export, sequestration, further reaction, or the like, depending on the particular application.

In some embodiments, the entrained flow design of the first reactor, as demonstrated in this example, may be used ensure substantially intimate mixing of the steam with the fuel or portions of the fuel, for instance, with volatile materials evolved from the fuel, so that the volatiles may be quantitatively converted to carbon monoxide and hydrogen, or other suitable products, within the first stripping reactor.

As shown in FIG. 2A, gas leaving the stripping reactor may be subjected to heat recovery, solids removal, desulfurization, further reaction, purification, etc., e.g., using commercially-available, known technologies. For instance, carbon monoxide and water produced may be converted to carbon dioxide and hydrogen via a gas shift reactor, which may be used to increase hydrogen production. As another example, carbon monoxide, which is a valuable synthetic reagent, may be exported to other processes such as the synthesis of methanol or ethanol. As yet another example, a portion of the hydrogen produced may be burned in the cogeneration plant and/or in an afterburner to generate electric power or to supply steam to the process. As still another example, some of the hydrogen may be reacted within the carbon export reactor, or stored for further use, e.g., as a hydrogen fuel.

For instance, sulfur that is captured upon exiting the first and/or second reactors can be converted to, e.g., solid elemental sulfur or sulfuric acid for sale, the choice depending upon factors such as process conditions or their relative economic value. For example, if high-sulfur fuels are being handled, the production of sulfuric acid can produce a significant amount of additional process heat in some cases, which may be used, for example, for heating steam. In this example, hydrogen is used to fuel the cogeneration plant to demonstrate the feasibility of approaching 100% sequestering of the global warming gases produced in both the combined gasification and cogeneration plant. It should be noted that many claims to 100% sequestration do not include the power requirements of the cogeneration plant (e.g., to produce steam), which often require the use of fossil fuels for power and the consequent release of gases such as CO or CO₂, or other global warming gases. In this example, in the carbon export reactor, the carbonaceous intermediate may be reacted with hydrogen (and/or other materials) to form one or more hydrocarbons, which can be exported from the process. The formation of hydrocarbons in this reactor can be an exothermic process so that little or no heat needs to be added in order to drive the reaction forward.

As shown in FIG. 2A, a portion of the hydrogen produced as described above may be passed through a plasma generator or “torch” in order to increase its reactivity. Hydrocarbons (or other compounds) produced in the second reactor may, in some cases, be subjected to further processing and/or reaction (for example, with more hydrogen) to provide an economically desirable product mix, depending on the specific components and the application. Solid residue containing ash, carbon, and/or other materials can also be discharged from the bottom of the carbon export reactor, and in some cases treated or sequestered. As shown in FIG. 2A, a portion of the carbon in the ash may be separated from the ash and recycled to the first reactor.

The process shown in this example may allow the processing of a wide array of fuels including fossil fuels such as coal or refinery coke, or renewable biomass fuels such as wood residue or bagasse. Thermal energy recovered from this process may be used, for example, for steam heating, to reduce the moisture content of some of these materials, etc. Typically, the fuel is pulverized or reduced in size before admission to the stripping reactor. Biomass and low rank coal may be useful in some embodiments due to their large content of hydrogen and oxygen, and these fuels may be highly reactive or contain a large amount of volatile materials in some cases. This allows processing under relatively mild thermal conditions, with consequent savings in energy. In addition, solid residues from biomass fuels may be produced in powdery form that can be used to return mineral nutrients to the soil, according to another embodiment of the invention.

The process in this example offers opportunities for improved energy efficiency and cost savings when compared to existing gasification technologies. For example, there may be a 50% to 70% by volume reduction in the required capacity of the air separation plant. The exact quantity of oxygen used for the process described in this example will depend on various factors, such as the composition of the feed stock, or the reaction rate economics. In addition, this process may also achieve some energy savings due to the conversion of fuel to carbon monoxide and/or hydrogen (e.g., syngas). This conversion requires that energy be added to drive the reaction, and this heat must be added at temperatures that often exceed 2000° F. The reactions of carbon monoxide and/or hydrogen to form hydrocarbons are exothermic, but these reactions typically occur at temperatures well below 1000° F. Because of the low temperature, the heat that is given off reduces the efficiency of power generation, due to thermodynamics. In general, and without wishing to be bound by any theory, it is believed that heat liberated at higher temperatures can therefore be converted to mechanical power more efficiently than heat liberated at low temperatures. Thus, this process may save energy by avoiding the conversion of all the carbon in the fuel to carbon monoxide and/or hydrogen. Instead, a portion of the carbon is used directly for hydrocarbon formation, e.g., within the second reactor.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
reacting a carbon-containing fuel with a gas comprising steam having a temperature of at least about 1500° F. in a first reactor to produce, at least, H₂ and a carbonaceous intermediate, wherein the carbonaceous intermediate is not fully oxidized; and
reacting the carbonaceous intermediate in a second reactor with H₂ to produce one or more hydrocarbons.

2. The method of claim 1, further comprising at least partially purifying the H₂.

3. The method of claim 1, further comprising reacting the H₂ with the carbonaceous intermediate.

4. The method of claim 1, further comprising reacting the H₂ to at least partially heat the gas comprising steam.

5. The method of claim 1, further comprising reacting at least a portion of the H₂ to produce a plasma.

6. The method of claim 1, wherein the gas comprising steam has a temperature of at least about 2000° F.

7. The method of claim 1, wherein the gas comprising steam has a temperature of at least about 2500° F.

8. The method of claim 1, wherein the gas comprising steam has a temperature of at least about 3000° F.

9. The method of claim 1, wherein the gas comprising steam has a temperature of at least about 3500° F.

10. The method of claim 1, wherein at least about 50 mol % of the carbon in the carbon-containing fuel is used to produce the carbonaceous intermediate.

11-13. (canceled)

14. The method of claim 1, wherein CO is produced in the first reactor.

15-55. (canceled)

56. The method of claim 54, wherein the ceramic heat exchanger comprises silicon carbide.

57. (canceled)

58. A method, comprising:
reacting a carbon-containing fuel in a first reactor to produce, at least, a carbonaceous intermediate, wherein at least about 50 mol % of the carbon in the carbon-containing fuel is used to produce the solid or liquid carbonaceous intermediate the first reactor further containing therein steam having a temperature of at least about 1500° F.;
reacting the carbonaceous intermediate in a second reactor to produce a hydrocarbon; and
at least partially purifying the hydrocarbon produced in the second reactor.

59-63. (canceled)

64. The method of claim 62, wherein the gas comprising steam is heated using a ceramic heat exchanger by combusting a portion of H₂ produced in the first reactor.

65-77. (canceled)

78. A method, comprising:
reacting a carbon-containing fuel with a gas comprising steam having a temperature of at least about 1500° F. to produce, at least, a carbonaceous intermediate, wherein the carbonaceous intermediate is not fully oxidized;
reacting the carbonaceous intermediate to produce a hydrocarbon; and
at least partially purifying the hydrocarbon.

79-82. (canceled)

83. The method of claim 78, wherein the gas comprising steam is heated using a ceramic heat exchanger.

84. (canceled)

85. The method of claim 78, wherein the act of reacting the carbon-containing fuel with the gas comprising steam and the act of reacting the carbonaceous intermediate to produce a hydrocarbon occur in different reactors.

86-120. (canceled)

121. The method of claim 14, further comprising reducing the CO with a water shift reaction to produce CO₂ and H₂.

122. The method of claim 54, wherein the ceramic heat exchanger is heated by heat produced by the combustion of H₂

123. The method of claim 54, wherein the ceramic heat exchanger heat exchanger comprises a composite.
